(19) United States
(12) Patent Application Publication
(10) Pub. No.: US 2016/0271258 A1
(43) Pub. Date: Sep. 22, 2016

(54) Dual Peptide-Mediated Targeted Delivery System

(71) Applicants: MUSC FOUNDATION FOR RESEARCH DEVELOPMENT, Charleston, SC (US); CLEMSON UNIVERSITY, Clemson, SC (US)

(72) Inventors: Andrew Jakymiw, Charleston, SC (US); Angela Alexander-Bryant, Charleston, SC (US)

(21) Appl. No.: 15/074,698

(22) Filed: Mar. 18, 2016

Related U.S. Application Data
(60) Provisional application No. 62/135,957, filed on Mar. 20, 2015.

(51) Int. Cl. A61K 47/42; C12N 15/113

(52) U.S. Cl. CPC A61K 47/42; C12N 15/113; C12N 2310/14

(57) ABSTRACT

This invention is generally related to a dual-peptide delivery system, where one peptide exhibits a receptor-targeting moiety and the other peptide exhibits an endosome-disruptive bioactivity.

### CROSS-REFERENCE TO RELATED APPLICATIONS

This application claims priority to U.S. Provisional Patent Application No. 62/135,957 filed Mar. 20, 2015, the contents of which are incorporated by reference herein in their entirety.

### STATEMENT REGARDING FEDERALLY SPONSORED RESEARCH OR DEVELOPMENT

This invention was made with government support under Grant Nos. NIDCR ROODE018191, NIGMS P30GM103331, NIDCR T32DE017551, and NIDCR R25DE022677 awarded by the National Institutes of Health (NIH). The government has certain rights in the invention.

### BACKGROUND OF THE INVENTION

Oral squamous cell carcinoma (OSCC) is a malignant neoplasm of the head and neck region accounting for over 90% of all subtypes of head and neck cancers. Cancer of the oral cavity and pharynx are a significant global burden with an incidence of 400,000 new cases and more than 200,000 deaths reported worldwide in 2008. In the USA, they represent 4% of the annually diagnosed malignancies in men and it is estimated that more than 45,000 Americans will be diagnosed and approximately 8,650 will die from cancer of the oral cavity or pharynx in 2015. Despite advances in the fields of oncology and surgery, the 5-year survival rate for all stages is approximately 66% and it has only modestly improved in the last 30 years.

RNA interference (RNAi) is a highly conserved post-transcriptional gene regulatory mechanism triggered by small, non-coding double-stranded RNA molecules that can specifically silence gene expression by either repressing translation and/or inducing mRNA degradation. The discovery that the introduction of chemically synthesized small interfering RNAs (siRNAs) into mammalian cells could efficiently induce sequence-specific inhibition of gene expression, made evident the therapeutic potential of harnessing RNAi as a means to specifically target and silence disease-causing genes. Although the design of therapeutic-grade siRNAs has improved, delivery still remains the single greatest obstacle towards the pervasive use of siRNAs for therapeutic applications. More specifically, cell/tissue-type specificity of siRNA delivery and endosomal entrapment are two of the major hurdles in RNAi therapeutics.

Despite advances made in the art, there remains a need in the art for improved systems enabling targeted delivery of a bioactive agent into a cell of interest with low toxicity. The present invention fulfills this need.

### SUMMARY OF THE INVENTION

The invention provides a composition comprising a dual peptide system for delivery of an agent. In one embodiment, the dual peptide system comprises 1) a first peptide comprising a targeting moiety and a stretch of densely packed cationic amino acid residues; 2) a second peptide having an endosome-disruptive activity and comprises a stretch of densely packed cationic amino acid residues.

In one embodiment, the dual peptide system comprises an agent. In one embodiment, the agent is a therapeutic agent. In one embodiment, the therapeutic agent is a nucleic acid molecule. In one embodiment, the therapeutic agent is selected from the group consisting of siRNA, microRNA, shRNA, antisense nucleic acid, ribozyme, killer-tRNAs, guide RNAs (part of the CRISPR/CAS system), long non-coding RNA, anti-miRNA oligonucleotide, and plasmid DNA.

In one embodiment, the stretch of densely packed cationic amino acid residues comprises at least nine cationic residues. In one embodiment, the at least nine cationic residues are arginines. In one embodiment, the arginine residues are D-arginine residues.

In one embodiment, the targeting moiety binds to a cell membrane receptor. In one embodiment, the cell membrane receptor is EGFR.

In one embodiment, the first peptide is SEQ ID NO:1.

In one embodiment, the second peptide is SEQ ID NO:2.

In one embodiment, the first peptide is SEQ ID NO:1 and the second peptide is SEQ ID NO:2, further wherein the first peptide, second peptide, and agent is at a ratio of 60:30:1.

In one embodiment, one or more of the peptides are further modified. In one embodiment, the modification is to enhance stability of the peptides. In one embodiment, the modification reduces renal clearance of the peptides. In one embodiment, the modification is polyethylene glycol (PEG).

The invention also provides a method of administering an agent into a cell with minimal cytotoxicity. In one embodiment, the method comprises contacting the cell with an effective amount of the dual peptide system of the invention.

The invention also provides a method of treating a disease or disorder in a subject in need thereof. In one embodiment, the method comprises administering to the subject a therapeutically effective amount of the dual peptide system of the invention.

In one embodiment, the subject is human.

In one embodiment, the disease or disorder is cancer.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic showing a model of EGFR-targeting endosome-disruptive dual peptide-mediated siRNA delivery.

FIG. 2 is a series of images demonstrating that the GE11R9+siCIP2A complex targets cells with high EGFR expression, but does not deliver bioactive siRNAs.

FIG. 3, comprising FIGS. 3A and 3B, is a series of images demonstrating that that the dual peptide retains the ability to specifically target cells with high EGFR expression.

FIG. 4 is an image demonstrating that dual peptide restores siCIP2A functionality in oral cancer cells.

FIG. 5 is an image demonstrating that both 599 and GE11R9 are necessary to achieve optimal CIP2A knockdown.

FIG. 6 is an image demonstrating that the 60:30:1 molar ratio induces a large and significant knockdown of CIP2A mRNA.

FIG. 7 is an image demonstrating that the dual peptide displays minimal cytotoxicity at molar ratios up to 60:30:1.

FIG. 8 is an image demonstrating that 599 and GE11R9 are able to completely complex siRNA at the 60:30:1 (GE11R9:599:siRNA) molar ratio.

FIG. 9 is an image demonstrating that 599 and GE11R9 protect siRNA from degradation in the presence of serum or RNase at the 60:30:1 molar ratio.

FIG. 10, comprising FIGS. 10A and 10B, depicts experimental results demonstrating that the dual-peptide-mediated technology significantly enhances the targeted delivery of complexed siRNAs to orthotopic oral cancer tumors 24 and 48 hours post-treatment. FIG. 10A depicts representative in vivo images of siRNA fluorescence intensity in whole animals at 1, 6, 24, and 48 hours post-treatment with Cy5.5-labeled siCIP2As complexed to either the dual-peptide (GE11R9+599) system or 599 peptide alone. FIG. 10B depicts quantitative analysis of siRNA fluorescence intensity in CAL 27-derived tumor tissues at 1, 6, 24, and 48 hours post-treatment. Data are mean ±SEM of three independent samples, where *P<0.05 and **P<0.01 compared to 599+siRNA complexes. Arrow, site of tumor; B/W, black and white image; A.U., arbitrary units.

### DETAILED DESCRIPTION OF THE INVENTION

The present invention is based on the discovery that a dual-peptide delivery strategy, where one peptide exhibits an endosome-disruptive bioactivity and the other exhibits a receptor-targeting moiety is able to synergistically deliver a bioactive agent into a desired cell. The invention also relates to methods of making and using such a dual peptide system to treat a variety of diseases and disorders.

### Definitions

As used herein, each of the following terms has the meaning associated with it in this section.

The articles “a” and “an” are used herein to refer to one or to more than one (i.e., to at least one) of the grammatical objects of the article. By way of example, “an element” means one element or more than one element.

“About” as used herein when referring to a measurable value such as an amount, a temporal duration, and the like, is meant to encompass variations of ±20% or ±10%, or ±5%, or ±1%, or ±0.1% from the specified value; as such variations are appropriate to perform the disclosed methods.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double-stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition.

An “anti-tumor effect” can also be manifested by the ability of the peptides, polynucleotides, cells and antibodies of the invention in prevention of the occurrence of tumor in the first place.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, oral cancer and the like. As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 36 nucleotides in length; for example, at least about 40 nucleotides to about 50 nucleotides; at least about 50 to about 60 nucleotides, at least about 60 to about 70 nucleotides; at least about 70 nucleotides to about 80 nucleotides; about 80 nucleotides to about 90 nucleotides; or about 100 nucleotides (and any integer value in between). As used herein, the term “fragment,” as applied to a protein or peptide, refers to a subsequence of a larger protein or peptide. A “fragment” of a protein or peptide can be at least about 12 amino acids in length; for example, a fragment of SEQ ID NO:1 can be at least about 12, 13, 14, 15, 16, 17, 18, 19, 20, 21, 22, 23, 24, or 25 amino acids in length.

The term “functionally equivalent” as used herein refers to a polypeptide according to the invention that preferably retains at least one biological function or activity of the specific amino acid sequence of either the first or second peptide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and mRNA stability, expression, function and activity, e.g., antagonists.

“Instructional material,” as that term is used herein, includes a publication, a recording, a diagram, or any other medium of expression which can be used to communicate the usefulness of the nucleic acid, polypeptide, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” when used herein refers to a detectable compound or composition that is conjugated directly or indirectly to a molecule to generate a “labeled” molecule. The label may be detectable by itself (e.g. radioisotope labels or fluorescent labels) or, in the case of an enzymatic label, may catalyze chemical alteration of a substrate compound or composition that is detectable (e.g., avidin-biotin).

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Individual miRNAs have been identified and sequenced in different organisms, and they have been given names. Names of miRNAs and their sequences are provided herein. Additionally, other miRNAs are known to those of skill in the art and can be readily implemented in embodiments of the invention. The methods and compositions should not be limited to miRNAs identified in the application, as they are provided as examples, not necessarily as limitations of the invention.

By the term “modulating,” as used herein, is meant mediating a detectable increase or decrease in the level of a mRNA, polypeptide, or a response in a subject, or a cell or tissue of a subject, as compared with the level of a mRNA, polypeptide or a response in the subject, or a cell or tissue of the subject, in the absence of a treatment or compound, and/or compared with the level of a mRNA, polypeptide, or a response in an otherwise identical, but untreated subject, or cell or tissue of the subject. The term encompasses perturbing and/or affecting a native signal or response thereby mediating a beneficial therapeutic response in a subject, preferably, a human.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982), which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including, but not limited to deletion, insertion, substitution of one or more nucleotides, or fusion to other polynucleotide sequences.

In another embodiment, the terms “ribonucleotide,” “oligoribonucleotide,” and “polyribonucleotide” refers to a string of at least 2 base-sugar-phosphate combinations. The term includes, in another embodiment, compounds comprising nucleotides in which the sugar moiety is ribose. In another embodiment, the term includes both RNA and RNA derivates in which the backbone is modified. “Nucleotides” refers, in another embodiment, to the monomeric units of nucleic acid polymers. RNA may be, in an other embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al., Genes & Devel 16: 2491-96 and references cited therein). In addition, these forms of RNA may be single, double, triple, or quadruple stranded. The term also includes, in another embodiment, artificial nucleic acids that may contain other types of backbones, but the same bases. In another embodiment, the artificial nucleic acid is a PNA (peptide nucleic acid). PNA contain peptide backbones and nucleotide bases and are able to bind, in another embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen PE, Curr Opin Struct Biol 9:353-57; and Raz NK et al. Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross-reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention generally relates to a dual peptide technology that can be used as a targeted carrier for the delivery of an agent into a desired cell. The dual peptide technology comprises a first peptide that exhibits a targeting moiety and a second peptide that exhibits an endosome-disruptive bioactivity, whereby the combination of both peptides allow for a synergistic delivery of an agent into a desired cell. Accordingly, the invention provides compositions and methods for delivery of an agent into a cell. In some instances, the dual peptide system of the invention can be used to deliver a therapeutic agent into a cell to treat a variety of diseases and disorders.

An advantage of the dual peptide system of the invention is that any targeting peptide can be combined with the endosome-disruptive peptide to deliver any agent into a desired cell, while keeping the endosome-disruptive peptide constant to treat different diseases. For example, the targeting moiety of the dual peptide system can be designed to mediate cell entry through receptor-mediated endocytosis initiated by binding of a ligand to a corresponding receptor. Upon entering the cell, the endosome-disruptive peptide component of the dual peptide system mediates endosomal escape of the agent into the cytoplasm. For example, the GE11R9 peptide mediates targeted cellular entry through receptor-mediated endocytosis initiated by binding of GE11R9 to EGFR. Upon entering the cell, 599 mediates endosomal escape of siRNA under acidic conditions and siRNA in the cytoplasm enters the RNA-induced silencing complex (RISC), which together enable sequence-specific inhibition of gene expression. In one embodiment, the dual peptide system of the invention comprising GE11R9, 599, and a desired siRNA exhibits minimal cytotoxicity and optimal therapeutic effects at ratios of about 60:30:1.

The dual peptide system of the invention may also have additional functions, such as the ability to bind to other proteins and agents. In certain embodiments, the invention includes methods of making such dual peptide system, methods of using the dual peptide system, kits involving such dual peptide system, and the like.

### Compositions

According to the present invention, the dual peptide system comprises a first peptide that exhibits a targeting function and a second peptide that exhibits an endosome-disruptive activity. In some instances one or more of the first and second peptide comprises a stretch of densely packed cationic amino acid residues to enable binding of a nucleic acid to the peptide via electrostatic interactions. In some instances, the stretch of densely packed cationic amino acid residues include nine arginines (R9). Preferably, the arginines are D-Arg.

In one embodiment, the targeting moiety allows for targeted delivery of the therapeutic agent (e.g., nucleic acid) into the desired cell and the endosome-disruptive activity of the second peptide mediates endosomal escape of therapeutic agent in the cytoplasm. In the situation where the therapeutic agent is a nucleic acid, for example siRNA, the siRNA enters the RNA-induced silencing complex (RISC), which together enable sequence-specific inhibition of gene expression.

The targeting moiety of the first peptide can be any molecule that is capable of specifically binding or interacting with a desired target.

In one embodiment, the targeting moiety can be any moiety recognized by a transmembrane or intracellular receptor protein.

In one embodiment, a targeting moiety is a ligand. The ligand, according to the present invention, preferentially binds to and/or internalizes into a cell in which the attached nucleic acid by way of the interaction with the densely packed cationic amino acid residues enters the cell. A ligand is usually a member of a binding pair where the second member is present on, or in a target cell, or in a tissue comprising the target cell. Examples of ligands suitable for the present invention are: folic acid, protein (e.g., transferrin), growth factor, enzyme, peptide, receptor, antibody or antibody fragment, such as Fab', Fv, single chain Fv, single-domain antibody, or any other polypeptide comprising antigen-binding sequences (CDRs) of an antibody molecule. In a preferred embodiment, the dual peptide system carrying a targeting moiety, e.g., a ligand, is internalized by a target cell. In yet another embodiment, a targeting moiety is a ligand that specifically interacts with a tyrosine kinase receptor such as, for example, EGFR, HER2, HER3, HER4, PD-GFR, VEGFR, bFGFR or IGFR receptors. In one embodiment, the targeting ligand specifically binds to Her2/neu markers. In still another embodiment, the targeting moiety specifically interacts with a growth factor receptor, an angiogenic factor receptor, a transferrin receptor, a cell adhesion molecule, or a vitamin receptor.

The choice of targeting moiety depends upon the type and number of ligands that define the surface of a target cell. For example, the targeting moiety may be chosen to recognize a ligand that acts as a cell surface marker on target cells associated with a particular disease state. Thus, examples of cell surface markers that may act as ligands for the targeting moiety in the dual peptide system of the invention include those associated with viral, bacterial and parasitic infections, autoimmune disease and cancer cells.

In one embodiment, the dual peptide system of the invention can be engineered to comprise a targeting moiety that targets a tumor antigen of interest by way of engineering a targeting moiety that specifically binds to an antigen on a tumor cell. In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response; particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include, but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens, such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature and unable to respond or they may be antigens that are normally present at extremely low levels on normal cells, but which are expressed at much higher levels on tumor cells.

The invention should not be limited to only the tumor antigen disclosed herein. Rather, the invention encompasses any cell surface marker where the dual peptide system of the invention can be engineered to have a targeting moiety that binds to the cell surface maker.

Depending on the desired antigen to be targeted, the dual peptide system of the invention can be engineered to include the appropriate antigen binding moiety that is specific to the desired antigen target. For example, if EGFR is the desired antigen that is to be targeted, a peptide that binds to EGFR (e.g., GE11) can be used as the antigen binding moiety for incorporation into the dual peptide system of the invention.

The dual peptide system of the invention can transport a desired agent into a variety of mammalian, amphibian, reptilian, avian, or insect cells. Cells can be primary cells or cell lines. Mammalian cells can be, e.g., human, monkey, rat, mouse, dog, cow, pig, horse, hamster, and rabbit. Primary cells from mammalians include, but are not limited to, adipocytes, astrocytes, cardiac muscle cells, chondrocytes, endothelial cells, epithelial cells, fibroblasts, gangliocytes, glandular cells, glial cells, hematopoietic cells, hepatocytes, keratinocytes, myoblasts, neural cells, osteoblasts, ovary cells, pancreatic beta cells, renal cells, smooth muscle cells, and striated muscle cells.

The peptides of the present invention further include conservative variants of the peptides herein described, according to another embodiment. As used herein, a “conservative variant” refers to alterations in the amino acid sequence that do not substantially and adversely affect the binding or association capacity of the peptide. A substitution, insertion or deletion is said to adversely affect the peptide when the altered sequence prevents, reduces, or disrupts a function or activity associated with the peptide.

For example, the overall charge, structure or hydrophobic-hydrophilic properties of the peptide can be altered without adversely affecting an activity. Accordingly, the amino acid sequence can be altered, for example to render the peptide more hydrophobic or hydrophilic, without adversely affecting the activities of the peptide.

These variants, though possessing a slightly different amino acid sequence than those recited elsewhere herein, will still have the same or similar properties associated with any of the peptides discussed herein. Ordinarily, the conservative substitution variants, will have an amino acid sequence having at least 75%, at least 80%, at least 85%, at least 90%, at least 95% amino acid, at least 98%, or at least 99% sequence identity with any of the peptides discussed elsewhere herein.

With respect to the stretch of densely packed cationic amino acid residues in the peptides of the invention, the peptide can comprise at least 4 cationic amino acid residues, such as arginine. In other embodiments, the peptide can comprise at least 5 cationic amino acid residues, such as arginine. In other embodiments, the peptide can comprise at least 6 cationic amino acid residues, such as arginine. In other embodiments, the peptide can comprise at least 7 cationic amino acid residues, such as arginine. In other embodiments, the peptide can comprise at least 8 cationic amino acid residues, such as arginine. In other embodiments, the peptide can comprise at least 9 cationic amino acid residues, such as arginine. Preferably, there is also a C-terminal Lysine residue after the cationic amino acid residues. More preferably, there is also a C-terminal Lysine residue after the 9 arginine residues The stretch of densely packed cationic amino acid residues is to enable binding of a nucleic acid to the peptide via electrostatic interactions.

Cationic properties can be determined, for instance, by using charge electrophoresis or isoelectric focusing to determine the charge of the protein (or portions of the protein) under various conditions. Any number of residues within the peptide may be arginine residues or the like, for instance, at least two residues, at least three residues, at least four residues, at least five residues, etc. In some cases, there may be between 3 and 6 or between 3 and 5 residues (inclusively) that are substituted with a cationic amino acid residue, such as arginine. The substitutions may be present on any location within the peptide, and may be consecutive or non-consecutive, in some cases.

In some embodiments, one or more components of the dual peptide delivery system of the invention are able to associate with (or bind to) specific sequences of DNA or other proteins. These proteins may be able to bind, for example, to DNA or other proteins with high affinity and selectivity. As used herein, the term “bind” or “binding” refers to the specific association or other specific interaction between two molecular species, such as, but not limited to, protein-DNA interactions and protein-protein interactions, for example, the specific association between proteins and their DNA targets, receptors and their ligands, enzymes and their substrates, etc. Such binding may be specific or non-specific, and can involve various noncovalent interactions, such as including hydrogen bonding, metal coordination, hydrophobic forces, van der Waals forces, pi-pi interactions, and/or electrostatic effects. It is contemplated that such association may be mediated through specific sites on each of two (or more) interacting molecular species. Binding can be mediated by structural and/or energetic components. In some cases, the latter will comprise the interaction of molecules with opposite charges.

The peptide of the present invention may be made using chemical methods. For example, peptides can be synthesized by solid phase techniques (Roberge J Y et at (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Peptide Analogs

The present invention relates to peptide analogs of GE11R9 (SEQ ID NO:1), 599 (SEQ ID NO:2) or another peptide appropriate for use with the invention and uses thereof. For example, in certain instances the invention provides peptides and peptide analogs based on fragments of GE11R9, wherein the peptides, including peptides and analogs, fragments, and derivatives thereof, of the invention exhibit desirable therapeutic properties. In one embodiment, the invention provides compositions comprising peptides and analogs, fragments, and derivatives thereof that exhibit one or more of improved solubility, half-life, bioavailability, reduced renal clearance and the like compared to full length GE11R9. In one embodiment, the invention provides compositions comprising peptides and analogs, fragments, and derivatives thereof that exhibit one or more of improved solubility, half-life, bioavailability, reduced renal clearance and the like compared to full length 599.

Fusion and Chimeric Polypeptides

A peptide of the invention may be conjugated with other molecules, such as proteins, to prepare fusion proteins or chimeric peptides. This may be accomplished, for example, by the synthesis of N-terminal or C-terminal fusion proteins provided that the resulting fusion protein or chimeric peptide retains the functionality of the peptide of the invention.

A peptide or chimeric protein of the invention may be phosphorylated using conventional methods such as the method described in Reedijk et al. (The EMBO Journal 11(4): 1365, 1992).

Cyclic derivatives of the peptides or chimeric proteins of the invention are also part of the present invention. Cyclization may allow the peptide or chimeric protein to assume a more favorable conformation for association with other molecules.

Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

In one embodiment, the subject compositions are peptidomimetics of the peptides of the invention, for example, peptidomimetics of the GE11R9 and/or 599 peptide. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

The peptidomimetics of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNALYS), could be modified with an amine specific photoaffinity label.

Nucleic Acid

The present invention further provides, in another embodiment, nucleic acid molecules that encode any of the amino acid sequences discussed herein. As used herein, “nucleic acid” includes cDNA and mRNA, as well as nucleic acids based on alternative backbones or including alternative bases whether derived from natural sources or synthesized. Those of ordinary skill in the art, given an amino acid sequence, will be able to generate corresponding nucleic acid sequences that can be used to generate the amino acid sequence, using no more than routine skill.

Modifications to the primary structure itself by deletion, addition, or alteration of the amino acids incorporated into the peptide sequence during translation can be made without destroying the activity of the peptide. Such substitutions or other alterations result in peptides having an amino acid sequence encoded by a nucleic acid falling within the contemplated scope of the present invention.

The present invention further provides, in some embodiments, recombinant DNA molecules that contain a coding sequence. As used herein, a “recombinant DNA molecule” is a DNA molecule that has been subjected to molecular manipulation. Methods for generating recombinant DNA molecules are well known in the art, for example, see Sambrook et al., 2012, Molecular Cloning: A Laboratory Manual, Cold Spring Harbor Laboratory, New York. In some recombinant DNA molecules, a coding DNA sequence is operably linked to expression control sequences and vector sequences.

The choice of vector and expression control sequences to which one of the peptide family encoding sequences of the present invention is operably linked depends directly, as is well known in the art, on the functional properties desired (e.g., protein expression, and the host cell to be transformed). A vector of the present invention may be at least capable of directing the replication or insertion into the host chromosome, and preferably also expression, of the structural gene included in the recombinant DNA molecule.

Expression control elements that are used for regulating the expression of an operably linked protein encoding sequence are known in the art and include, but are not limited to, inducible promoters, constitutive promoters, secretion signals, and other regulatory elements. In some embodiments, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extra-chromosomal in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical of bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Any suitable prokaryotic host can be used to express a recombinant DNA molecule encoding a peptide of the invention.

Expression vectors compatible with eukaryotic cells, including those compatible with vertebrate cells, can also be used to form recombinant DNA molecules that contain a coding sequence. Eukaryotic cell expression vectors are well known in the art and are available from several commercial sources. Typically, such vectors are provided containing convenient restriction sites for insertion of the desired DNA segment.

Eukaryotic cell expression vectors used to construct the recombinant DNA molecules of the present invention may further include a selectable marker that is effective in a eukaryotic cell, such as a drug resistance selection marker. An example drug resistance marker is the gene whose expression results in neomycin resistance, i.e., the neomycin phosphotransferase (neo) gene. Alternatively, the selectable marker can be present on a separate plasmid, the two vectors introduced by co-transfection of the host cell, and transfectants selected by culturing in the appropriate drug for the selectable marker.

The present invention further provides, in yet another embodiment, host cells transformed with a nucleic acid molecule that encodes a peptide of the present invention. The host cell can be either prokaryotic or eukaryotic. Eukaryotic cells useful for expression of a peptide of the invention are not limited, so long as the cell line is compatible with cell culture methods and compatible with the propagation of the expression vector and expression of the gene product.

Transformation of appropriate cell hosts with a recombinant DNA molecule encoding a peptide of the present invention is accomplished by well-known methods that typically depend on the type of vector used and host system employed. With regard to transformation of prokaryotic host cells, electroporation and salt treatment methods can be employed (see, for example, Sambrook et al., 2012, Molecular Cloning: A Laboratory Manual, Cold Spring Harbor Laboratory Press). With regard to transformation of vertebrate cells with vectors containing recombinant DNA, electroporation, cationic lipid or salt treatment methods can be employed (see, for example, Graham et al., (1973) Virology 52, 456-467; Wigler et al., (1979) Proc. Natl. Acad. Sci. USA 76, 1373-1376).

Successfully transformed cells can be identified by well-known techniques including the selection for a selectable marker. For example, cells resulting from the introduction of a recombinant DNA of the present invention can be cloned to produce single colonies. Cells from those colonies can be harvested, lysed and their DNA content examined for the presence of the recombinant DNA using a method such as that described by Southern (1975) J. MoI. Biol. 98, 503-517, or the peptides produced from the cell assayed via an immunological method.

The present invention further provides, in still another embodiment, methods for producing a peptide of the invention using nucleic acid molecules herein described. In general terms, the production of a recombinant form of a peptide typically involves the following steps: a nucleic acid molecule is obtained that encodes a peptide of the invention.

The nucleic acid molecule may then be placed in operable linkage with suitable control sequences, as described above, to form an expression unit containing the peptide open reading frame. The expression unit is used to transform a suitable host and the transformed host is cultured under conditions that allow the production of the recombinant peptide. Optionally the recombinant peptide is isolated from the medium or from the cells; recovery and purification of the peptide may not be necessary in some instances where some impurities may be tolerated.

Each of the foregoing steps can be done in a variety of ways. The construction of expression vectors that are operable in a variety of hosts is accomplished using appropriate replicons and control sequences, as set forth above. The control sequences, expression vectors, and transformation methods are dependent on the type of host cell used to express the gene. Suitable restriction sites, if not normally available, can be added to the ends of the coding sequence, so as to provide an excisable gene to insert into these vectors. An artisan of ordinary skill in the art can readily adapt any host/expression system known in the art for use with the nucleic acid molecules of the invention to produce a recombinant peptide.

In another embodiment, the present invention provides methods for use in isolating and identifying binding partners of the peptides of the invention. In some embodiments, a peptide of the invention is mixed with a potential binding partner or an extract or fraction of a cell under conditions that allow the association of potential binding partners with the peptide of the invention. After mixing, peptides, polypeptides, proteins or other molecules that have become associated with a peptide of the invention are separated from the mixture. The binding partner bound to the peptide of the invention can then be removed and further analyzed. To identify and isolate a binding partner, the entire peptide can be used. Alternatively, a fragment of the peptide which contains the binding domain can be used.

In another embodiment, the nucleic acid molecules encoding a peptide of the invention can be used in a yeast two-hybrid system. The yeast two-hybrid system has been used to identify other peptide partner pairs and can readily be adapted to employ the nucleic acid molecules herein described (see, e.g., Stratagene Hybrizap® two-hybrid sy stem).

According to some embodiments, the peptides of the invention are useful for drug screening to identify agents capable of binding to the same binding site as the peptides. The peptides are also useful for diagnostic purposes to identify the presence and/or detect the levels of DNA or protein that binds to the peptides of the invention. In one diagnostic embodiment, the peptides of the invention are included in a kit used to detect the presence of a particular DNA or protein in a biological sample. The peptides of the invention also have therapeutic uses in the treatment of disease associated with the presence of a particular DNA or protein. In one therapeutic embodiment, the peptides can be used to bind to DNA to promote or inhibit transcription, while in another therapeutic embodiment, the proteins bind to a protein resulting in inhibition or stimulation of the protein.

In some embodiments of the invention, peptides of the invention are administered to a subject in an effective amount to treat a cancer or a disease or disorder.

The peptides of the invention may be administered to cells of a subject to treat or prevent a disease or disorder (e.g., cancers) alone or in combination with the administration of other therapeutic compounds for the treatment or prevention of these diseases or disorders.

In certain embodiments, the peptides of the invention are useful for diagnostic purposes to identify the presence and/or detect the levels of a target protein that binds to the proteins of the invention. The peptides of this method can be labeled with a detectable marker. A wide range of detectable markers can be used, including but not limited to biotin, a fluorogen, an enzyme, an epitope, a chromogen, or a radionuclide. In one embodiment, the detectable marker can be conjugated to the C-terminal Lysine of the peptide. The method for detecting the label will depend on the nature of the label and can be any known in the art, e.g., film to detect a radionuclide, an enzyme substrate that gives rise to a detectable signal to detect the presence of an enzyme, antibody to detect the presence of an epitope, etc.

### Therapeutic Agent

In certain embodiments, the composition of the present invention comprises a therapeutic agent which is delivered to a cell or tissue of interest via the dual peptide technology described elsewhere herein. In certain embodiments, the therapeutic agent interacts with the stretch of densely packed cationic amino acid residues of one or both of the first and second peptide. For example, in one embodiment, the therapeutic agent interacts with the R9 of one or both of the first and second peptide.

RNA interference (RNAi) is normally triggered by double stranded RNA (d5RNA) or endogenous microRNA precursors (pri-miRNAs/pre-miRNAs). Since its discovery, RNAi has emerged as a powerful genetic tool for suppressing gene expression in mammalian cells. Stable gene knockdown can be achieved by expression of synthetic short hairpin RNAs (shRNAs). In one embodiment, the therapeutic agent comprises a nucleic acid molecule. The nucleic acid molecule may be DNA, RNA, cDNA, microRNA, siRNA, shRNA, or the like.

An siRNA polynucleotide is an RNA nucleic acid molecule that interferes with RNA activity that is generally considered to occur via a post-transcriptional gene silencing mechanism. An siRNA polynucleotide preferably comprises a double-stranded RNA (d5RNA), but is not intended to be so limited and may comprise a single-stranded RNA (see, e.g., Martinez et al., 2002 Cell 110:563-74). The siRNA polynucleotide included in the invention may comprise other naturally occurring, recombinant, or synthetic single-stranded or double-stranded polymers of nucleotides (ribonucleotides or deoxyribonucleotides or a combination of both) and/or nucleotide analogues as provided herein (e.g., an oligonucleotide or polynucleotide or the like, typically in 5′ to 3′ phosphodiester linkage). Accordingly it will be appreciated that certain exemplary sequences disclosed herein as DNA sequences capable of directing the transcription of the siRNA polynucleotides are also intended to describe the corresponding RNA sequences and their complements, given the well-established principles of complementary nucleotide base-pairing.

Preferred siRNA polynucleotides comprise double-stranded polynucleotides of about 18-30 nucleotide base pairs, preferably about 18, about 19, about 20, about 21, about 22, about 23, about 24, about 25, about 26, or about 27 base pairs, and in other preferred embodiments about 19, about 20, about 21, about 22 or about 23 base pairs, or about 27 base pairs, whereby the use of “about” indicates that in certain embodiments and under certain conditions the processive cleavage steps that may give rise to functional siRNA polynucleotides that are capable of interfering with expression of a selected polypeptide may not be absolutely efficient. Hence, siRNA polynucleotides, may include one or more siRNA polynucleotide molecules that may differ (e.g., by nucleotide insertion or deletion) in length by one, two, three, four or more base pairs as a consequence of the variability in processing, in biosynthesis, or in artificial synthesis of the siRNA. The siRNA polynucleotide of the present invention may also comprise a polynucleotide sequence that exhibits variability by differing (e.g., by nucleotide substitution, including transition or transversion) at one, two, three or four nucleotides from a particular sequence. These differences can occur at any of the nucleotide positions of a particular siRNA polynucleotide sequence, depending on the length of the molecule, whether situated in a sense or in an antisense strand of the double-stranded polynucleotide. The nucleotide difference may be found on one strand of a double-stranded polynucleotide, where the complementary nucleotide with which the substitute nucleotide would typically form hydrogen bond base pairing, may not necessarily be correspondingly substituted. In preferred embodiments, the siRNA polynucleotides are homogeneous with respect to a specific nucleotide sequence.

Based on the present disclosure, it should be appreciated that the siRNAs of the present invention may effect silencing of the target polypeptide expression to different degrees. The siRNAs thus must first be tested for their effectiveness. Selection of siRNAs are made therefrom based on the ability of a given siRNA to interfere with or modulate the expression of the target polypeptide. Accordingly, identification of specific siRNA polynucleotide sequences that are capable of interfering with expression of a desired target polypeptide requires production and testing of each siRNA. The methods for testing each siRNA and selection of suitable siRNAs for use in the present invention are fully set forth herein the Examples. Since not all siRNAs that interfere with protein expression will have a physiologically important effect, the present disclosure also sets forth various physiologically relevant assays for determining whether the levels of interference with target protein expression using the siRNAs of the invention have clinically relevant significance.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention.

In a preferred embodiment, the modulating sequence is an antisense nucleic acid sequence which is expressed by a plasmid vector. The antisense expressing vector is used to transfect a mammalian cell or the mammal itself, thereby causing reduced endogenous expression of a desired regulator in the cell. However, the invention should not be construed to be limited to inhibiting expression of a regulator by transfection of cells with antisense molecules. Rather, the invention encompasses other methods known in the art for inhibiting expression or activity of a protein in the cell including, but not limited to, the use of a ribozyme, the expression of a non-functional regulator (i.e. transdominant negative mutant) and use of an intracellular antibody.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of a regulator may be designed by incorporating target sequences into the basic ribozyme structure which are complementary to the mRNA sequence of the desired target of the present invention. Ribozymes targeting the desired regulator may be synthesized using commercially available reagents (Applied Biosystems, Inc., Foster City, Calif.) or they may be genetically expressed from DNA encoding them.

In some embodiments of the invention, a miRNA or a synthetic miRNA is used as a therapeutic agent to regulate gene expression. The miRNA may contain one or more design elements. These design elements include, but are not limited to: i) a replacement group for the phosphate or hydroxyl of the nucleotide at the 5′ terminus of the complementary region; ii) one or more sugar modifications in the first or last 1 to 6 residues of the complementary region; or, iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′ oxygen-methyl), DMTO (4,4′-dimethoxytrityl with oxygen), fluoroscein, a thiol, or acridine, though other replacement groups are well known to those of skill in the art and can be used as well.

Additional embodiments concern a synthetic miRNA having one or more sugar modifications in the first or last 1 to 6 residues of the complementary region (referred to as the “sugar replacement design”). In certain cases, there are one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, that have a sugar modification. It will be understood that the terms “first” and “last” are with respect to the order of residues from the 5′ end to the 3′ end of the region. In particular embodiments, the sugar modification is a 2′O-Me modification. In further embodiments, there are one or more sugar modifications in the first or last 2 to 4 residues of the complementary region or the first or last 4 to 6 residues of the complementary region.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

In one embodiment, the nucleic acid therapeutic agent is a DNA, RNA, cDNA molecule, or the like which encodes a therapeutic peptide or protein. For example, in certain embodiments, once the nucleic acid enters a desired cell, via the dual peptide delivery system, the nucleic acid molecule may be transcribed and/or translated into a desired therapeutic peptide.

### Modification

There exist negative charges on the renal glomerular basement membrane consisting of patches of proteoglycan. These proteoglycan molecules can interact with cationic peptide molecules. Further, cationic peptides are more readily deposited in the glomerular basement membrane than a more anionic peptide. A consequence of rapid renal clearance associated with the deposition of cationic peptide molecules onto the glomerular basement membrane is a rapid serum clearance and decreased serum half-life. Similarly, there may exist other clearance mechanisms which are able to recognize charged molecules and more rapidly effect excretion resulting in a reduced half-life of the peptides of the invention. Therefore, the peptides of the invention may be modified to produce variants, analogs and fragments of the proteins which have a reduced rate of clearance or increased half-life, so long as the desired biological properties (i.e., the ability to bind to the target site or the ability to have the same effector activity as the parent unmodified peptide) is retained. The peptides may be modified by using various genetic engineering or protein engineering techniques.

In one embodiment, a peptide of the invention is further modified. In one embodiment, a functional fragment of a peptide of the invention contains a further modification. In one example, a further modification of a peptide includes a modification at the N-terminus. In one embodiment a further modification comprises a modification at the C-terminus. In one embodiment a peptide of the invention is modified at both the N- and C-termini.

In one embodiment, a modification of a peptide of the invention is a chemical modification, conjugation to a synthetic or natural polymer, glycosylation, acetylation, methylation, phosphorylation, conjugation of a chemical linker, fatty acyl derivatization, or conjugation of a polysaccharide. In one embodiment, modification of a peptide of the invention includes fusion to human serum albumin (HAS) or an albumin-binding peptide or protein. In one embodiment, modification of a peptide of the invention includes linkage to an immunoglobin Fc domain to form an Fc fusion protein. In one embodiment, a polymer to be conjugated to a peptide on the invention is one of polyethylene glycol (PEG), polypropylene glycol (PPG), polysialic acid (PSA), XTEN™ and hydroxyethyl starch (HES). In one exemplary embodiment, PEGylation of one or more peptides of the invention can increase its half-life by retarding renal clearance, as the PEG moiety adds hydrodynamic radius to the peptide. Conventional PEGylation methodologies directed to monomeric proteins are well known in the art.

In one embodiment, a single polymer is conjugated to a single peptide of the invention. In one embodiment, multiple polymers are conjugated to a single peptide of the invention. In one embodiment, a population of peptides of the invention will contain a mixture of modified and unmodified peptides having 0 to more than 1 polymer conjugated to each peptide.

In one embodiment, an additional peptide is provided for use with the dual peptide targeting system. In one embodiment, an additional peptide is anionic. In one embodiment, an additional peptide contains a further modification. In one embodiment, a further modification of an additional peptide is PEGylation. In one exemplary embodiment, a third PEGylated anionic peptide serves to coat and mask the cationic charge of the complex formed from a mixture of the dual peptide system with an agent.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

### Pharmaceutical Compositions and Methods of Treatment

In various embodiments, the present invention is a method of treating a disease or disorder in a subject in need thereof, by administering to the subject, one or more components of the dual peptide delivery system of the invention as described elsewhere herein.

In one embodiment, the dual peptide system of the invention is administered with a desired agent at a molar ratio that exhibits effective binding and specific uptake of the agent into the cell. The ratio can be optimized in order to synergistically mediate the effective targeting and delivery of the agent into the cell and result in optimal targeting with minimal cytotoxicity.

In one embodiment, a 1:1:1 ratio of the first peptide that exhibits a targeting moiety (1^{st} peptide), the second peptide that exhibits an endosome-disruptive bioactivity (2^{nd} peptide), and the agent is used. In certain aspects of the present invention, a ratio of 1^{st} peptide: 2^{nd} peptide: agent is used, such that an increase in targeting a cell, endosome-disruptive bioactivity, and activity of the agent is observed as compared to the effects observed using a ratio of 1:1:1. In one particular embodiment an increase of from about 1 to about 3 fold is observed as compared to the activity of the agent observed using a ratio of 1:1:1. In one embodiment, the ratio of 1^{st} peptide: 2^{nd} peptide: agent ranges from 100:1:1 to 1:100:1 and all integer values there between. In one aspect of the present invention, more of the 1^{st} peptide is used compared to the 2^{nd} peptide. In certain embodiments of the invention, the ratio of 1^{st} peptide: 2^{nd} peptide: agent is about 100:10:1; about 100:20:1; about 100:30:1; about 100:40:1; about 100:50:1; about 100:60:1; about 100:70:1; about 100:80:1; about 100:90:1; about 100:100:1. In certain embodiments of the invention, the ratio of 1^{st} peptide: 2^{nd} peptide: agent is about 10:100:1; about 20:100:1; about 30:100:1; about 40:100:1; about 50:100:1; about 60:100:1; about 70:100:1; about 80:100:1; about 90:100:1; about 100:100:1. In certain embodiments, the ratio of 1^{st} peptide: 2^{nd} peptide: agent is about 60:30:1.

As those of ordinary skill in the art can readily appreciate, the ratio of 1^{st} peptide: 2^{nd} peptide: agent may depend on the target moiety of the first peptide. The ratio of 1^{st} peptide: 2^{nd} peptide: agent may also depend on the agent. One of skill in the art will appreciate that a variety of other ratios may be suitable for use in the present invention.

In one embodiment, the fraction of a 1^{st} peptide in the 1^{st} peptide: 2^{nd} peptide: agent composition comprises unmodified 1^{st} peptide. In one embodiment, the fraction of a 1^{st} peptide in the 1^{st} peptide: 2^{nd} peptide: agent composition comprises modified 1^{st} peptide. In one embodiment, the fraction of a 1^{st} peptide in the 1^{st} peptide: 2^{nd} peptide: agent mixture comprises a mixture of modified and unmodified 1^{st} peptide.

In one embodiment, the fraction of a 2^{nd} peptide in the 1^{st} peptide: 2^{nd} peptide: agent composition comprises unmodified 2^{nd} peptide. In one embodiment, the fraction of a 2^{nd} peptide in the 1^{st} peptide: 2^{nd} peptide: agent composition comprises modified 2^{nd} peptide. In one embodiment, the fraction of a 2nd peptide in the 1^{st} peptide: 2^{nd} peptide: agent mixture comprises a mixture of modified and unmodified 2^{nd} peptide.

It will be appreciated that the peptides of the invention may be administered to a subject either alone, or in conjunction with another therapeutic agent. In one embodiment, the peptides of the invention are administered to a subject in combination with an anti-cancer therapy.

The therapeutic and prophylactic methods of the invention thus encompass the use of pharmaceutical compositions comprising one or more components of the dual peptide delivery system of the invention to practice the methods of the invention. The pharmaceutical compositions useful for practicing the invention may be administered to deliver a dose of from 100 ng/kg/day to 100 mg/kg/day. In one embodiment, the invention envisions administration of a dose which results in a concentration of the compound of the present invention from 1 μM to 10 μM in a mammal.

Typically, dosages which may be administered in a method of the invention to a mammal, preferably a human, range in amount from 0.5 μg to about 50 mg per kilogram of body weight of the mammal. The precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Dual Peptide-Mediated Targeted Delivery of siRNAs into Cancer Cells in Vitro

Two major hurdles for small interfering RNA (siRNA)-mediated therapies are cell/tissue-type targeted delivery and endosomal entrapment of siRNAs, resulting in inefficient gene silencing. Experiments were designed to examine the feasibility of utilizing two peptides, one with cancer cell-targeting specificity and the second with endosome-disruptive properties, to co-deliver bioactive siRNAs into oral cancer cells overexpressing the epidermal growth factor receptor (EGFR) and induce silencing of the targeted oncoprotein, CIP2A. In order to induce targeted uptake, the EGFR targeting peptide, GE11R9, was designed to target delivery of siRNAs to EGFR-overexpressing oral cancer cells. The GE11R9 peptide alone was found to deliver siRNAs specifically to EGFR-overexpressing oral cancer cells; however, it was not capable of mediating the delivery of bioactive siRNAs. This data indicated that the GE11R9 peptide was mediating internalization of the siRNAs via receptor mediated endocytosis, but that the siRNAs were incapable of escaping endosomes. Interestingly, co-addition of the endosome-disruptive peptide, 599, to a mixture of GE11R9 and siRNAs at a 60:30:1 (GE11R9:599:siRNA) molar ratio exhibited effective binding and specific uptake of siRNAs into EGFR-overexpressing cells compared to low-EGFR-expressing cells. Most importantly, the co-addition of the 599 peptide at the 60:30:1 molar ratio restored siRNA functionality. Together, these data demonstrate that this dual-peptide strategy can synergistically mediate the effective targeting and delivery of siRNAs into EGFR-overexpressing cancer cells and result in silencing of the targeted oncogene.

The results of this example are now described.

Model of Dual Peptide-Mediated siRNA Delivery Both the GE11R9 and 599 peptides complex with siRNA using a stretch of densely packed cationic nona(D-arginine) amino acid residues (R9) which enable complexing via electrostatic interactions.

The sequence of GE11 is disclosed in Li et al. (2005 FASEB J, 19:1978-1985). GE11R9 was generated by modifying the GEl 1 sequence to include a stretch of densely packed cationic amino acid residues (R9) to enable binding of siRNAs to the peptide via electrostatic interactions. The GE11R9 peptide also comprises a C-terminal Lysine. The GE11R9 peptide can also be engineered to comprise a detectable label (e.g., biotin).

The sequence of the 599 peptide is disclosed in Cantini et al. (2013 PLoS ONE, 8(9):e733458). The 599 peptide contains a derivative sequence of influenza virus protein with endosome-disruptive properties (INF7) and includes a stretch of densely packed cationic amino acid residues (R9) to enable binding of siRNAs to the peptide via electrostatic interactions. The 599 peptide also comprises a C-terminal Lysine. The 599 peptide can also be engineered to comprise a detectable label (e.g., biotin).

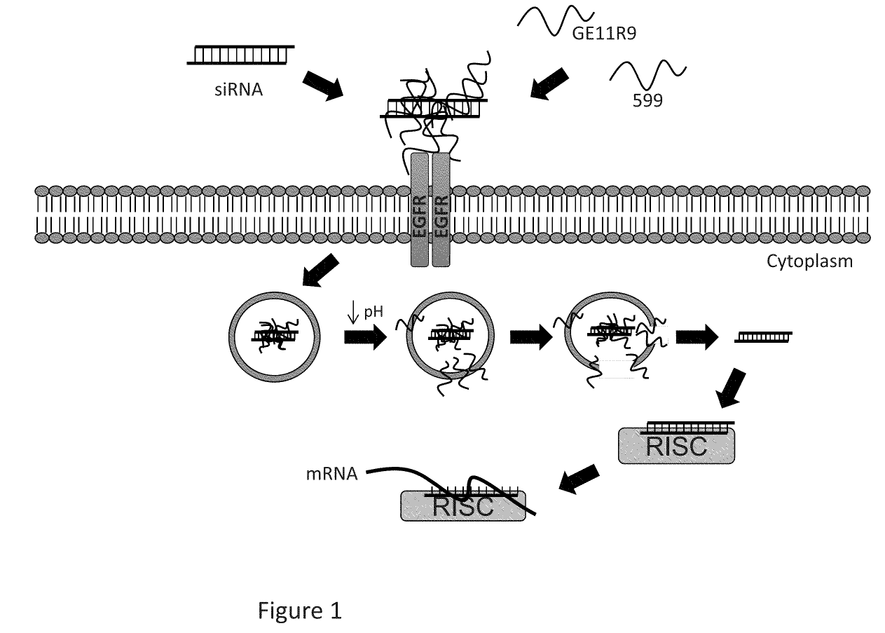

The GE11R9 peptide mediates cell entry through receptor-mediated endocytosis initiated by binding of GE11R9 to EGFR. Upon entering the cell, 599 mediates endosomal escape of siRNA under acidic conditions and siRNA in the cytoplasm enters the RNA-induced silencing complex (RISC), which together enable sequence-specific inhibition of gene expression (FIG. 1).

GE11R9+siCIP2A Complex Targets Cells with High EGFR Expression, but does not Deliver Bioactive siRNAs

Fluorescence microscopy analysis was performed after co-culture of CAL 27 (high EGFR) and SCC-15 (low EGFR) oral cancer cells incubated for 4 hours with 100 nM of DY547-conjugated siRNA targeting CIP2A (D-siCIP2A; red) in complex with GE11R9 peptide at 100:1 and 60:1 (GE11R9:siRNA) molar ratios. SCC-15 cells were stained for vimentin (green) and nuclei (blue) were counterstained with DAPI.

Western blot analysis of CIP2A protein expression levels in CAL 27 OSCC cells was performed 48 hours post-treatment with GE11R9 peptide complexed to either 100 nM of siNT or siCIP2A at a 60:1 or 100:1 peptide-to-siRNA molar ratio compared to 599 peptide at a 50:1 molar ratio. β-Actin protein levels were monitored to ensure equal loading of samples.

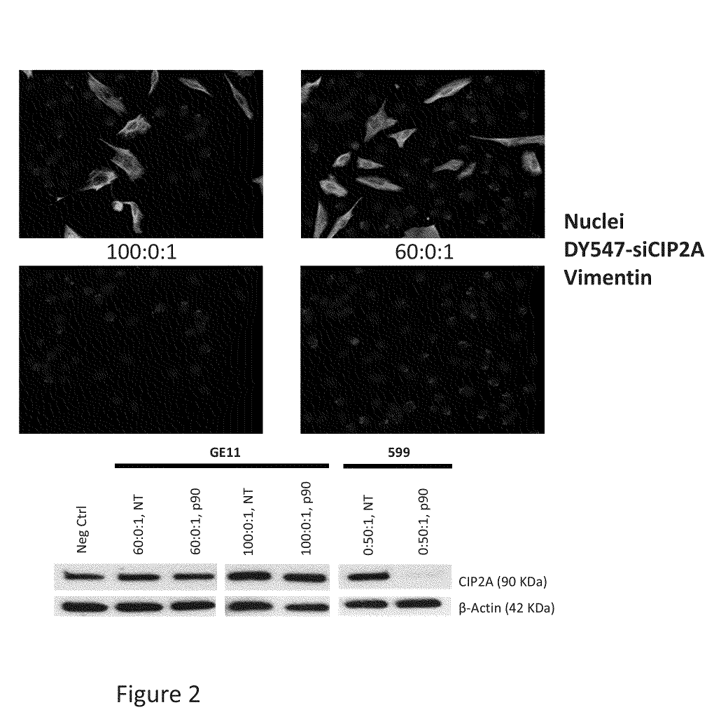

FIG. 2 demonstrates that the GE11R9+siCIP2A complex targets cells with high EGFR expression, but does not deliver bioactive siRNAs.

Dual peptide retains the ability to specifically target cells with high EGFR expression

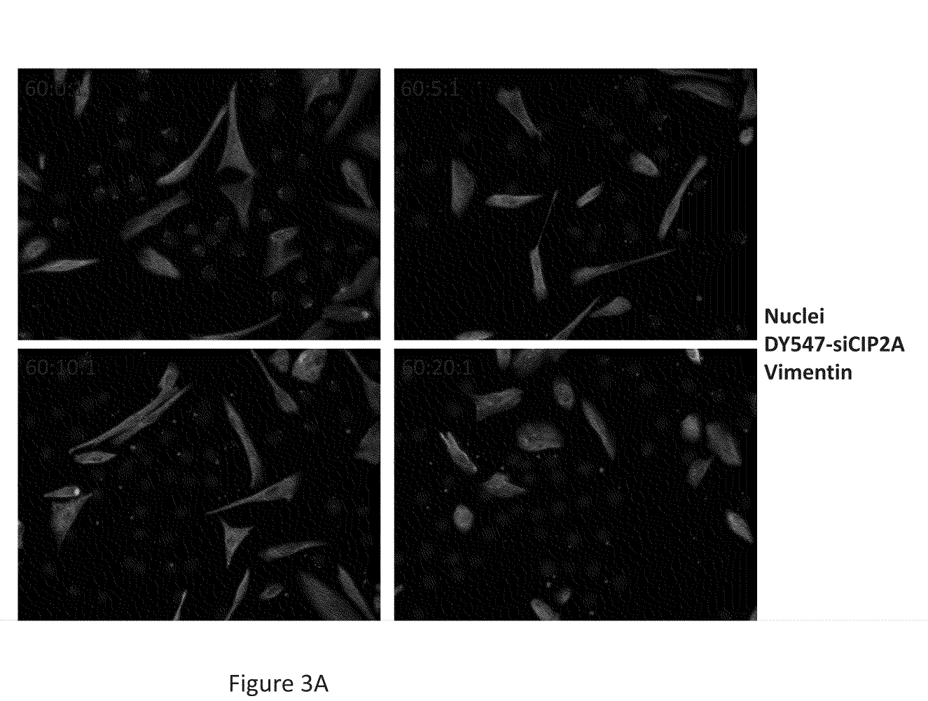

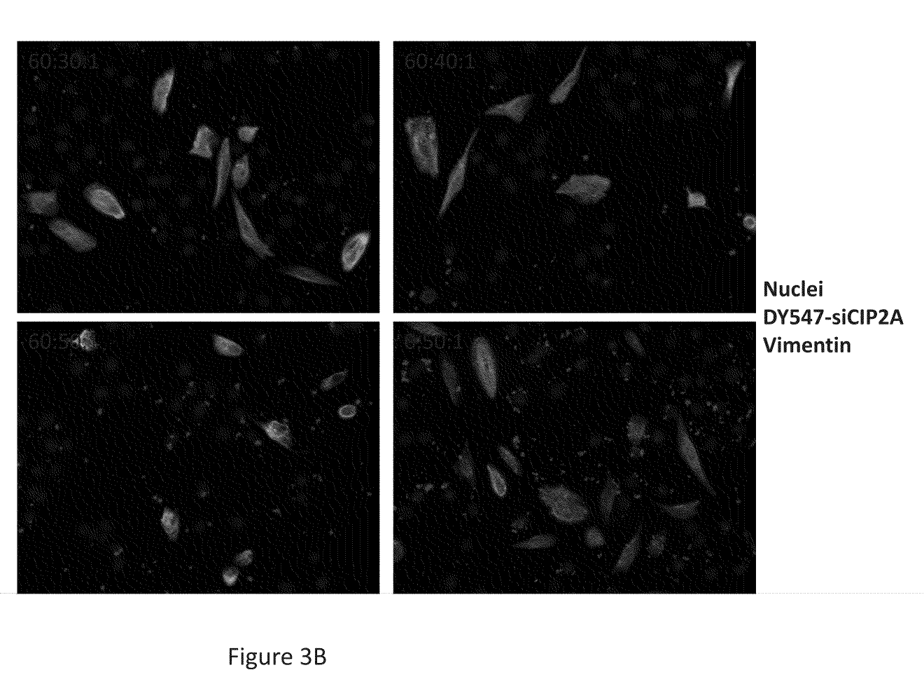

Fluorescence microscopy analysis of the co-culture of CAL 27 (high EGFR) and SCC-15 (low EGFR) oral cancer cells incubated for 4 hours with 100 nM of DY547-conjugated siRNA targeting CIP2A (D-siCIP2A; red) in complex with GE11R9 and 599 peptides at various molar ratios (FIGS. 3A & 3B). SCC-15 cells were stained for vimentin (green) and nuclei (blue) were counterstained with DAPI

Dual Peptide Restores siCIP2A Functionality in Oral Cancer Cells

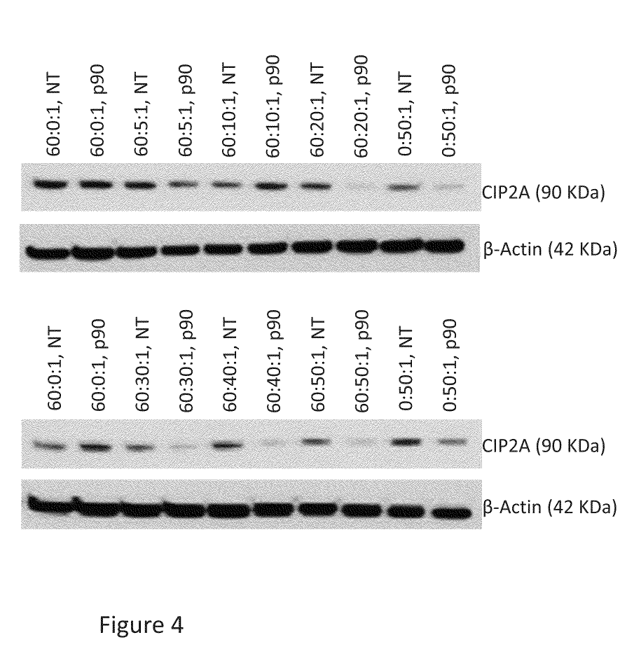

FIG. 4 is a Western blot analysis of CIP2A protein expression levels in CAL 27 OSCC cells performed 48 hours post-treatment with dual peptide complexed to either 100 nM of siNT or siCIP2A at various GE11R9:599:siRNA molar ratios. β-Actin protein levels were monitored to ensure equal loading of samples.

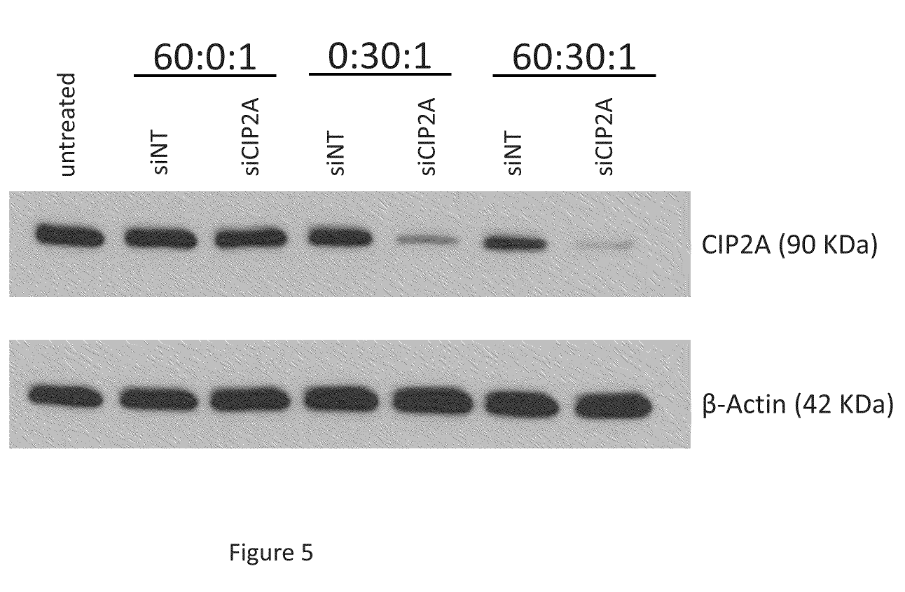

Both 599 and GE11R9 are necessary to achieve optimal CIP2A knockdown (FIG. 5).

The 60:30:1 (GE11R9:599:siRNA) Molar Ratio Demonstrates a Large and Significant Knockdown of CIP2A mRNA

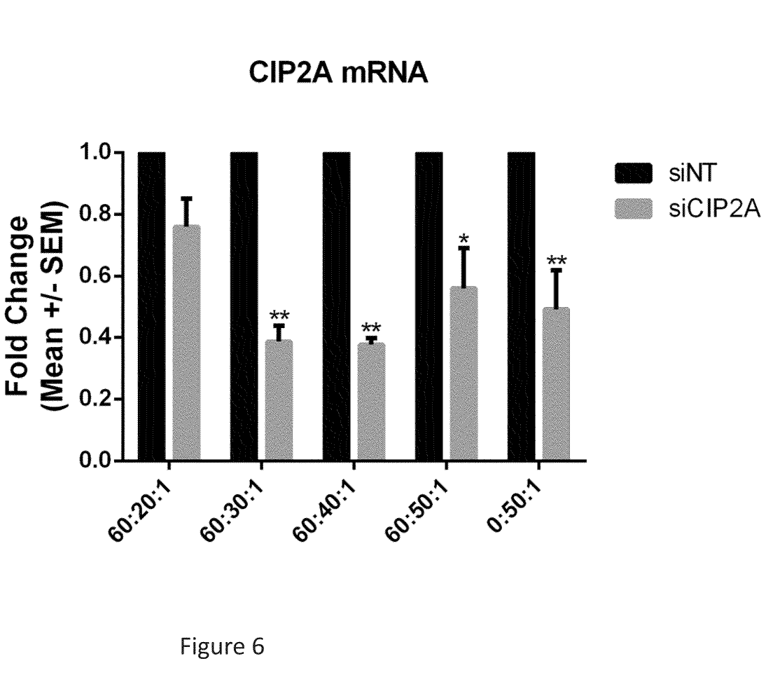

FIG. 6 shows that the 60:30:1 (GE11R9:599:siRNA) molar ratio demonstrates optimal knockdown of CIP2A mRNA. Real-time PCR analysis of CIP2A mRNA levels in CAL 27 oral cancer cells was performed 48 hours post-treatment with the dual peptide complex at various GE11R9:599:siRNA molar ratios using 100 nM of siRNA targeting CIP2A (siCIP2A) compared with control non-targeting siRNA (siNT). The CIP2A mRNA levels were normalized to 18S rRNA. Data are mean ±SEM of three independent experiments performed in triplicate, where *P<0.05 and **P<0.01 compared to siNT treated cells (Student's t test).

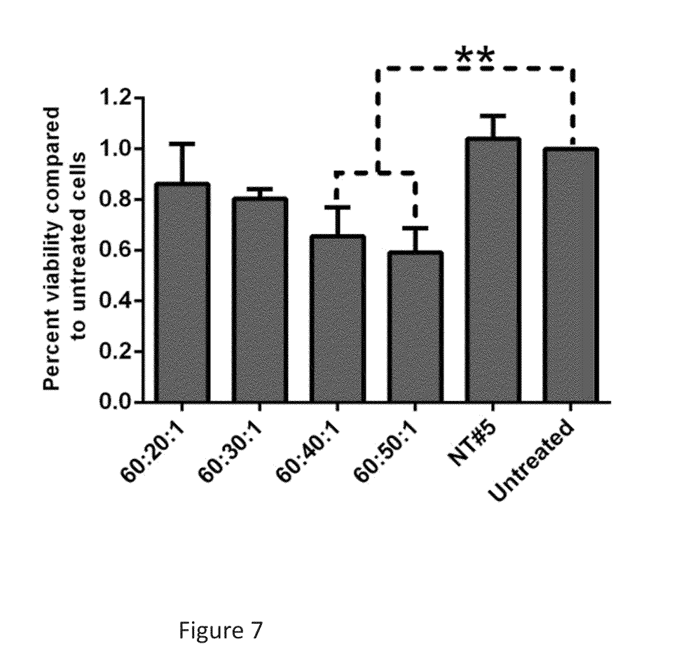

The dual peptide displays minimal cytotoxicity at ratios up to 60:30:1 (FIG. 7).

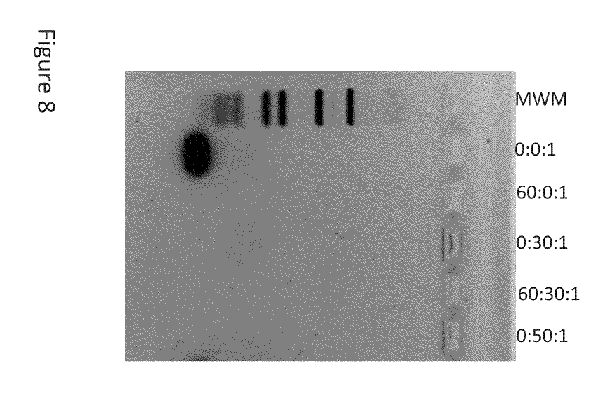

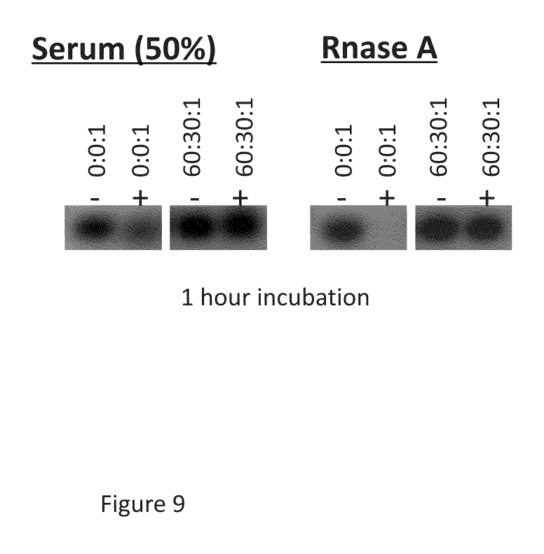

599 and GE11R9 are able to completely complex siRNA at the 60:30:1 ratio (FIG. 8). 599 and GE11R9 protect siRNA from degradation in the presence of serum or RNase at 60:30:1 ratio (FIG. 9).

The results presented herein demonstrate that 599 alone delivers bioactive siCIP2A in vitro and in vivo, but is not cell specific. GE11R9 alone specifically targets

EGFR overexpressing cells, but does not deliver bioactive siRNAs. However, the dual peptide complex, combining GE11R9 with 599 and siCIP2A retains the ability to specifically target EGFR-overexpressing cells and restores bioactivity. At a 60:30:1 (GE11R9:599:siCIP2A) molar ratio, the dual peptide complex exhibits optimal knockdown of CIP2A mRNA with minimal cytotoxicity.

For therapy, the dual peptide strategy could be used to more efficiently deliver therapeutic siRNAs to diseased cells/tissues. Although the results presented herein are directed to oral cancer, these peptides can equally be used to deliver any small double-stranded RNAs (dsRNAs) (e.g. siRNAs and/or microRNAs) to any diseased tissue of choice by modifying the targeting peptide. Also for basic research applications, the dual peptide can be used as a transfection agent to deliver small dsRNAs into cells grown in tissue culture.

Further experiments can be designed to investigate the ability of the dual peptide-siRNA complex to target delivery of siRNAs to cancer tissue and silence gene expression in vivo. For example, a xenograft tumor floor-of-mouth mouse model can be treated by systemically administering the dual peptide-siRNA complex via tail vein injection. The optimal concentration for delivery, biodistribution, half-life, and bioactivity can be determined. Subsequently, experiments can be designed to assess whether the complex triggers inflammation and/or an immune response in immunocompetent mice and examine whether multiple dosings of the complex impair tumor growth and promote survivability of the animal.

The dual peptide technology can therefore be used as a targeted carrier for the delivery of nucleic acids to diseased tissues. An advantage of this system is that one could potentially interchange the targeting peptide and targeting siRNA while keeping the endosome-disruptive peptide constant to treat different diseases.

### Example 2: Dual Peptide-Mediated Targeted Delivery of siRNAs into Mice

A dual peptide-mediated target delivery system has been designed to utilize two peptides to co-deliver small interfering RNAs (siRNAs) into cancer cells that overexpress the epidermal growth factor receptor (EGFR), and consequently induce silencing of the targeted oncoprotein, CIP2A. One peptide possesses cancer cell-targeting specificity, and the other, endosome-disruptive properties. Previous efforts produced a novel endosome-disruptive cell-penetrating peptide, termed 599, that was demonstrated to mediate intracellular delivery of bioactive siRNAs targeting CIP2A (siCIP2A). Subsequent studies have demonstrated that the 599 peptide can protect siRNAs from degradation upon intratumoral injection in vivo and induce CIP2A silencing, consequently resulting in the significant inhibition of tumor growth. In order to induce targeted uptake of siRNAs, the inventors also designed an EGFR-targeting peptide termed, GE11R9. GE11R9 was shown to deliver siRNAs specifically to EGFR-overexpressing cancer cells; however, it was not able to mediate the delivery of bioactive siRNAs. Consequently, the endosome-disruptive peptide, 599, and the EGFR-targeting peptide, GE11R9, were co-complexed with siRNAs in a dual-peptide strategy at a specified molar ratio, which resulted in effective binding and specific uptake of siRNAs into EGFR-overexpressing cancer cells, compared to low-EGFR-expressing cells. Most importantly, this combination restored siRNA functionality. Together, these data suggest that the dual peptide strategy can synergistically mediate the effective targeting and delivery of siRNAs into EGFR-overexpressing cancers cells, and result in silencing of the targeted oncogene.

Evidence for the dual-peptide system's application to basic research is provided through in vitro data demonstrating the dual-peptide's ability to complex with siRNAs, protect siRNAs from degradation by serum and ribonucleases, target the delivery of siRNAs to cancer cells, and silence the target gene of interest with no significant toxicities.

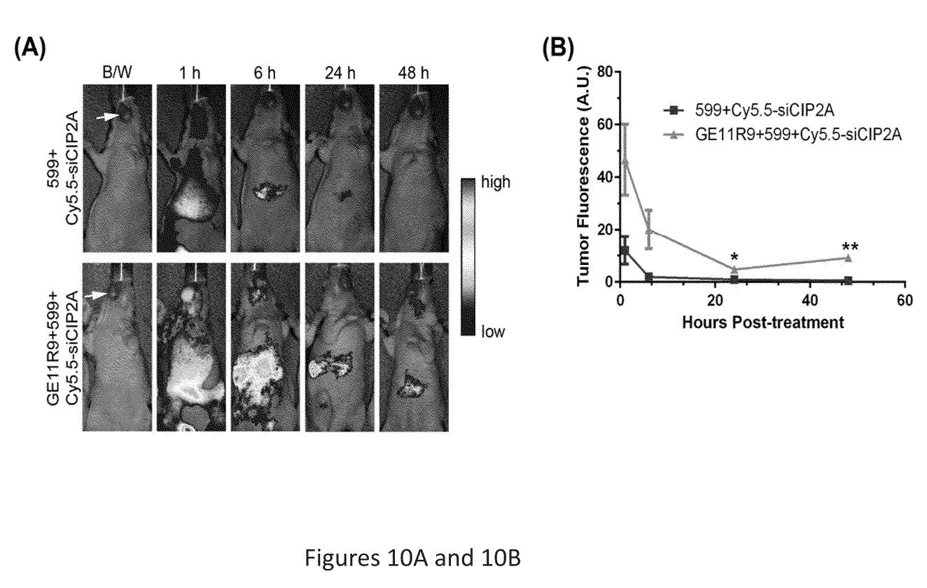

Recent studies have shown through in vivo bioimaging analyses that, when administered systemically via tail-vein injections into mice bearing orthotopic xenograft human oral cancer tumors, the dual-peptide system was found to mediate increased targeted delivery of siRNAs into the tumor tissues in comparison to 599 peptide alone (FIG. 10).

A non-limiting example of the application of this dual-peptide strategy includes clinical use to deliver any small double-stranded RNAs (dsRNAs) (e.g. siRNAs and/or microRNAs) to a diseased tissue of choice through a modification of the targeting peptide. A non-limiting example of the application to basic research includes use as a transfection agent to deliver small dsRNAs or other nucleic acid molecules into cells grown in tissue culture.

Advantages of the dual-peptide strategy include: (1) ease of use as the peptides for use in the system are charged and therefore are readily soluble in water and to generate the complex, one only needs to mix the peptides with the siRNAs at the specified peptide1: peptide2:siRNA molar ratio; (2) very efficient targeted delivery of small non-coding RNAs (e.g. siRNAs) into human cancer cells overexpressing EGFR in vitro; (3) enhanced targeting and delivery of siRNAs to orthotopic xenograft human tumors in mice in vivo following systemic administration; (4) high levels of silencing of the target gene at both the mRNA and protein levels in vitro; (5) no significant effects on long term cell viability in vitro (48 hours post treatment); (6) therapeutic potential; and (7) low cost of peptide synthesis.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a dual peptide system for delivery of an agent comprising: 1) a first peptide comprising a targeting moiety and a stretch of densely packed cationic amino acid residues; 2) a second peptide having an endosome-disruptive activity and comprising a stretch of densely packed cationic amino acid residues; and 3) an agent.

2. The composition of claim 1, wherein the agent is a therapeutic agent.

3. The composition of claim 2, wherein the therapeutic agent is a nucleic acid molecule.

4. The composition of claim 2, wherein the therapeutic agent is selected from the group consisting of siRNA, microRNA, shRNA, antisense nucleic acid, ribozyme, killer-tRNAs, guide RNAs (part of the CRISPR/CAS system), long non-coding RNA, anti-miRNA oligonucleotide, and plasmid DNA.

5. The composition of claim 1, wherein the stretch of densely packed cationic amino acid residues comprises at least nine cationic residues.

6. The composition of claim 5, wherein the at least nine cationic residues are arginine residues.

7. The composition of claim 5, wherein the at least nine cationic residues are D-arginine residues.

8. The composition of claim 1, wherein the targeting moiety binds to a cell membrane receptor.

9. The composition of claim 8, wherein the cell membrane receptor is EGFR.

10. The composition of claim 1, wherein the first peptide is SEQ ID NO:1.

11. The composition of claim 1, wherein the second peptide is SEQ ID NO:2.

12. The composition of claim 1, wherein the first peptide is SEQ ID NO:1 and the second peptide is SEQ ID NO:2, further wherein the first peptide, second peptide, and agent is at a ratio of 60:30:1.

13. The composition of claim 1, wherein one or more peptides further comprise an additional modification to reduce renal clearance.

14. The composition of claim 13, wherein the additional modification is polyethylene glycol (PEG).

15. The composition of claim 1, further comprising one or more additional peptides.

16. The composition of claim 15, wherein the one or more additional peptides are anionic.

17. The composition of claim 15, wherein the one or more additional peptides comprise an additional modification.

18. The composition of claim 17, wherein the additional modification is polyethylene glycol (PEG).

19. A method of administering an agent into a cell with minimal cytotoxicity, the method comprising contacting the cell with an effective amount of the composition of claim 1.

20. A method of treating a disease or disorder in a subject in need thereof, the method comprising administering to the subject a therapeutically effective amount of the composition of claim 1.

21. The method of claim 20, wherein the subject is human.

22. The method of claim 20, wherein the disease or disorder is cancer.


SEQUENCE LISTING (2 sequences)

SEQ ID NO: 1 - protein, 26 aa, Artificial Sequence
  GE11R9
YHWYGYTPQNVIGGGGRRRRRRRRRK

SEQ ID NO: 2 - protein, 36 aa, Artificial Sequence
  599 Peptide
GLFEAIEGFIENGWEGMIDGWYGGGGRRRRRRRRRK